(19) United States
(12) Patent Application Publication
(10) Pub. No.: US 2022/0257330 A1
(43) Pub. Date: Aug. 18, 2022

(54) HAND-OPERATED MEDICAL INSTRUMENT WITH APPARATUS FOR DETECTING ACTIVATION CYCLES

(71) Applicant: Aesculap AG, Tuttlingen (DE)

(72) Inventors: Roland-Alois Hoegerle, Tuttlingen (DE); André Buerk, Villingen-Schwenningen (DE); Thomas-Erwin Kahler, Seitingen-Oberflacht (DE); Frederick Lenzenhuber, Tuttlingen (DE)

(21) Appl. No.: 17/625,869

(22) PCT Filed: Jul. 10, 2020
(86) PCT No.: PCT/EP2020/069614

§ 371 Date: Jan. 10, 2022

(30) Foreign Application Priority Data
Jul. 11, 2019 (DE) 10 2019 118 869.5

(51) Int. Cl. A61B 90/00; G01R 31/00

(52) U.S. Cl. CPC A61B 90/08; A61B 2090/0803; G01R 31/00

(57) ABSTRACT

A medical instrument includes an apparatus for detecting activation cycles of the instrument. First and second parts of the instrument are movable relative to each other in an activation cycle. An energy detection coil detects inductively generated energy. An energy-generating magnet inductively generates energy to be detected by the energy detection coil. In the event of movement of the first movable part and the second movable part relative to each other, the energy-generating magnet is movable in a detection region of the energy detection coil and induces energy. A memory and processing device has a counter, detects a voltage signal based on the induced energy during each relative movement, increases the counter by one for each detection of the voltage signal, and counts the number of activation cycles of the instrument. In an alternative embodiment, the voltage signal is generated using a voltage created by a piezoelectric element.

### CROSS-REFERENCES TO RELATED APPLICATIONS

This application is the United States national phase entry of International Application No. PCT/EP2020/069614, filed Jul. 10, 2020, and claims priority to German Application No. 10 2019 118 869.5, filed Jul. 11, 2019. The contents of International Application No. PCT/EP2020/069614 and German Application No. 10 2019 118 869.5 are incorporated by reference herein in their entireties.

### FIELD

The invention relates to an apparatus and a system for detecting activation cycles of a hand-operated instrument, and in particular relates to an apparatus and a system for detecting, counting and/or documenting activation cycles of instruments in the medical field, such as scissors, clamps, punches, laparoscopic instruments and the like, and for providing relevant information obtained therefrom for further processing.

### BACKGROUND

Up to now, it has not been possible to detect and document activation cycles in hand-operated, unpowered instruments from the medical field in electronic and/or digital form and to further process relevant findings obtained on this basis.

Due to unavailable information such as such activation cycles, it is also not possible to make at least partially automated statements about the general condition of a hand-operated product or instrument, about a service life that can still be expected or an end of service life that has already 30 been reached for the same, its performance capability and its suitability for subsequent operations (as described, for example, in a purpose statement) and/or an existing overload and any damage that may have occurred.

Up to now, tests and visual inspections have had to be carried out for this purpose, which are time-consuming and cost-intensive and, due to the lack of detected data, are also subject to a range of assessment in each case, which, with the application of safety margins, may lead to products and instruments being discarded as a precaution (and thus too early), even though they could possibly still be used for a longer period of time if they were assessed on the basis of detected and, in this respect, secured usage data. This in turn leads to increased procurement efforts with associated costs and to the disposal of more material than would be necessary in accordance with an actual state of use.

Furthermore, due to a lack of corresponding data, it is not possible to create customized services and individually tailored business models for the customer, which is also not optimal for the customer in terms of procurement and operating costs.

In addition, issues relating to service life and the reasoning and evidence in cases of complaints, for example, are becoming increasingly important.

### SUMMARY

The object of the invention is to provide an apparatus and a system for detecting activation cycles of a hand-operated, unpowered product or instrument in the medical field, via which a user or customer can directly detect the number of times the unpowered product or instrument has already been used, i.e. can count how many times it has already been operated and whether it can still perform its predetermined function.

The number of activation cycles is generally a proportional measure of, among other things, the general condition of an instrument, its service life or end of service life, its performance 30 capability and suitability for an upcoming operation (as described in the intended purpose), and its overload and any product damage that may have occurred. Another measure may be reprocessing cycles of the instrument, which may be determined via a readable data memory such as an RFID chip, via NFC and BLE and the like, in conjunction with a suitable data acquisition system.

The invention is based on the general idea of providing an apparatus working as activation-cycle counter for detecting activation cycles of an unpowered, hand-operated, medical instrument.

The aforementioned activation-cycle counter is arranged to count actuations of the instrument and, in conjunction with an RFID/NFC/BLE module, to provide a means for indicating a remaining service life of the instrument. The detection is based on a sensor device operating without external power supply, wherein the detectable or countable and further processable actuation signal is generated from a hand movement of the user, for example a surgeon, during the use of the instrument.

The basic idea is that a passing magnet (neodymium (iron-boron), samarium-cobalt, etc.) is induced with energy via a coil and each one-time of passing of the magnet is detected. Alternatively, this basic idea also includes that when the instrument is actuated, pressure is applied via a pressure-generating element disposed thereon to a piezo element also disposed on the instrument, which then generates a voltage, and each one-time voltage generation is detected. Each of the aforementioned actions increases a value in a counter by 1. The respective current counter reading can then be read out at any time via RFID/NFC peripherals (e.g. smartphones and the like) using a combination of, for example, a chip and a core plus a coil, which represents a current design of RFID/NFC chips.

In each case, the apparatus in the form of an assembly group ‘activation-cycle counter with integrated coil for detecting movement via magnet’ is surrounded by a closed housing (e.g. made of glass, a ceramic, injected in a plastic part, integrated in a plastic part, etc.), which protects internal electronics against processing media and temperature. Readout of data is performed via an additional or external apparatus with a corresponding receiving device (for example, a smartphone, a smart tray, a tablet, and the like). Read-out data can be written to an external data memory (for example, a cloud service, a database, and the like). The solution according to the invention is transferable and scalable to all items, products and/or instruments that have a certain two-part structure.

It is understood that, in accordance with the shape of a respective instrument and/or an application of the instrument, combinations of magnets, coils, pressure-generating elements and piezo elements with corresponding signal conversion and also, if applicable, the omission of individual ones of the aforementioned elements are conceivable, as long as in the end a countable signal suitable for counting each activation cycle is generated.

It is further understood that the invention is in no way limited to the medical field and products, systems and/or instruments used there, but correspondingly modified configurations and modifications for numerous further and/or other products are conceivable and representable.

In detail, the object is solved by an apparatus for detecting activation cycles of a hand-operated instrument with at least a first and a second movable part, wherein in an activation cycle the first and the second movable part are movable relative to each other. The apparatus includes an energy detection coil arranged on one of the first and second movable parts and arranged to detect inductively generated energy; an energy-generating magnet arranged on the other one of the first and second movable parts and arranged to inductively generate energy to be detected by the energy detection coil, wherein the energy-generating magnet and the energy detection coil are arranged such that, upon movement of the first and second movable parts relative to each other, the energy-generating magnet is movable within a detection range of the energy detection coil relative thereto and induces energy therein via the energy detection coil; and a memory and processing device having a counter and arranged to detect, at each relative movement, a voltage signal based on the energy induced in the energy detection coil and to increment the counter by one at each detection of the voltage signal, thereby counting the number of executed activation cycles of the instrument.

In a special embodiment, the instrument is not supplied with energy, i.e. it is designed as an instrument that is autarkic regarding an external energy source (with internal energy conversion device). In other words, in particular, the instrument is not powered by an external energy source. In other words, the instrument is supplied with energy exclusively via internal energy conversion devices. In other words, the instrument has here no physical connection, in particular no cable to an external energy source, i.e. outside the instrument.

In another special embodiment, the instrument may be powered in a mixed way by both an internal smoothing buffer module with charging electronics and by manual actuation of the two relatively movable parts. That is, the smoothing buffer module and the manual actuation can act as internal energy sources.

In an alternative embodiment, the instrument may be powered solely by manual actuation of the two relatively movable parts.

Preferably, a magnetic core is arranged in the apparatus and the energy detection coil is wound on the magnetic core. The magnetic core with its associated coil is used in conjunction with a corresponding chip or combined component to provide RFID/NFC functionality, among other things. By winding the energy detection coil directly onto the magnetic core, a separate energy detection coil can advantageously be omitted.

Preferably, a magnetic core is arranged in the apparatus, the energy detection coil is arranged separately from the magnetic core and the magnetic core has a further coil wound onto it. Dedicated coils on the magnetic core and for the detection advantageously increase the degree of freedom in adapting the apparatus to a particular intended application.

Preferably, the energy-generating magnet is a neodymium or a samarium-cobalt magnet and is surrounded by a shield in such a way that its magnetic field is directed in an effective direction towards the energy detection coil and is attenuated in directions other than the effective direction.

Advantageously, only one predeterminable effective direction is achieved.

Preferably, the energy-generating magnet is insertable into the energy detection coil and induces energy therein by a linear movement. Such an arrangement is advantageous in certain applications.

Preferably, the energy-generating magnet that can be inserted into the energy detection coil is arranged on a pivot arm and can be caused to oscillate by a body mechanically acting on the pivot arm. Advantageously, in this way the energy-generating magnet can generate prolonged energy.

Preferably, the energy-generating magnet is rotatably arranged in the energy detection coil and induces energy therein by a rotary movement. Furthermore, a transmission gearing may be arranged to adjust the rotational speed of the rotary movement, and/or a flywheel mass may be arranged to assist in maintaining the rotary movement.

Alternatively, the object is solved in detail by an apparatus for detecting activation cycles of a hand-operated instrument with at least a first and a second movable part, wherein in an activation cycle the first and the second movable part are movable relative to each other. The device includes a piezoelectric element arranged on one of the first and second movable parts and arranged to be subjected to pressure, tension, and/or torsion, and to thereby create a voltage; an electronic circuit assembly for operating the piezoelectric element; a pressure-generating element arranged on the other one of the first and second movable parts and arranged to subject the piezoelectric element to pressure, tension, and/or torsion; an energy detection coil arranged to convert the voltage generated by the piezoelectric element into a detectable voltage signal, wherein the piezoelectric element and the pressure-generating element are arranged in such a way that upon movement of the first and the 20 second movable parts relative to each other, the pressure-generating element applies the pressure, the tension, and/or the torsion to the piezoelectric element and the voltage thereby generated by the piezoelectric element is applied to the energy detection coil and there generates the detectable voltage signal; and a memory and processing device comprising a counter and being arranged to detect the voltage signal generated in the energy detection coil at each relative movement and to increment the counter by one at each detection of the voltage signal, thereby counting the number of executed activation cycles of the instrument.

Preferably, a magnetic core is also arranged in this alternative apparatus, wherein the magnetic core has a coil wound on it. The magnetic core with its associated coil serves in conjunction with a corresponding chip or combined component, among other things, to provide RFID/NFC functionality. By winding the energy detection coil directly onto the magnetic core, a separate energy detection coil can be advantageously omitted. However, an energy detection coil may also be arranged separately from the magnetic core and the magnetic core may have another coil wound onto it. Dedicated coils on the magnetic core and for detection advantageously increase the degrees of freedom in adapting the apparatus to a particular intended application.

Preferably, the memory and processing device comprises an EEPROM and an integrated circuit or is formed as a combination component, and in conjunction with the magnetic core and the coil wound thereon provides an externally addressable and/or readable RFID/NFC apparatus with RFID/NFC functionality.

Preferably, the apparatus includes a smoothing buffer module with charging electronics, wherein the smoothing buffer module comprises a capacitor, a PowerCap™ brand buffer module, and/or a battery and is provided to support the memory and processing device. Advantageously, the assembly group is extended by an additional smoothing buffer module (capacitor, PowerCap™ brand buffer module, battery, etc.) with charging electronics, for example to keep the chip or combined component receivable for a longer time.

Preferably, a pivot joint connecting the first movable part and the second movable part consists at least partially of a piezo element arranged to energize the apparatus via pressure and torsion.

Preferably, the piezo element is in the form of a housing and is arranged to tightly enclose the other components of the apparatus and to induce energy in the apparatus when subjected to vibration, shock or pressure.

### BRIEF DESCRIPTION OF THE DRAWINGS

The invention is described in more detail below with reference to the accompanying drawings, of which:

FIG. 1 shows a schematic representation of an apparatus for detecting activation cycles of a hand-operated instrument according to a first configuration example;

FIG. 2 shows a schematic representation of an apparatus for detecting activation cycles of a hand-operated instrument according to a second configuration example;

FIG. 3 shows a schematic representation of an apparatus for detecting activation cycles of a hand-operated instrument according to a third configuration example;

FIG. 4 shows a schematic representation of an apparatus for detecting activation cycles of a hand-operated instrument according to a fourth configuration example;

FIG. 5 shows a schematic representation of a magnet surrounded by a shield, usable as an energy-generating magnet in configuration examples;

FIG. 6 shows a schematic representation of an apparatus for detecting activation cycles of a hand-operated instrument according to a fifth configuration example;

FIG. 7 shows a schematic representation of an apparatus for detecting activation cycles of a 20 hand-operated instrument according to a sixth configuration example;

FIG. 8 shows a schematic representation of an apparatus for detecting activation cycles of a hand-operated instrument according to a seventh configuration example;

FIG. 9 shows a schematic representation of an apparatus for detecting activation cycles of a hand-operated instrument according to an eighth configuration example;

FIG. 10 shows a schematic representation of an apparatus for detecting activation cycles of a hand-operated instrument in an alternative embodiment according to a ninth configuration example;

FIG. 11 shows a schematic representation of a piezo element usable in configuration examples with associated power management; and

FIG. 12 shows a schematic representation of an apparatus for detecting activation cycles of a hand-operated instrument according to a tenth configuration example.

In the figures, identical reference signs denote identical or at least equivalent parts and components. Expediently, a redundant, repeated description of such parts and components is omitted in this respect.

### DETAILED DESCRIPTION

Preferred configuration examples of an apparatus described herein for detecting, counting, and/or documenting activation cycles of a hand-operated, unpowered product or instrument (hereinafter also referred to as an activation-cycle counter) are described below with reference to the accompanying figures.

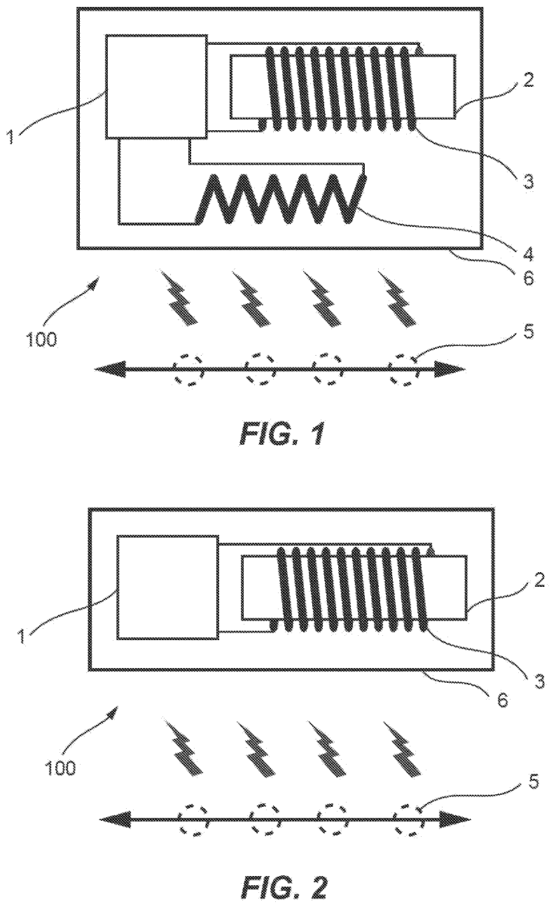

FIG. 1 shows a schematic representation of the apparatus for detecting activation cycles of a hand-operated instrument or activation-cycle counter 100 according to a first configuration example.

According to FIG. 1, the activation-cycle counter 100 includes a memory and processing device 1 that may be provided in the form of an EEPROM, an IC having an integrated circuit assembly, a chip, or a combination component having one or more of the aforementioned elements and arranged generally to store and process data, values, signals, etc. detected, processed, or 25 otherwise provided in the activation-cycle counter. Furthermore, the activation-cycle counter 100 includes a magnetic core 2 having a coil assembly or coil 3 wound directly thereon. The coil 3 is connected at each of its two ends to respective inputs on the memory and processing device 1. In addition, the activation-cycle counter 100 has an energy detection coil 4 for providing a signal, for example a voltage signal, which is also connected at its ends to corresponding inputs of the memory 30 and processing device 1 in order to implement a counting function in the memory and processing device 1. In the first configuration example, the energy detection coil 4 is arranged separately from the coil 3 of the magnetic core 2. A tightly sealing housing 6 surrounds the aforementioned components or assembly groups 2 to 4 of the activation-cycle counter 100.

In addition, the activation-cycle counter 100 includes an energy-generating magnet 5 outside the housing 6 which is movable relative to the energy detection coil 4 and may be made of, for example, neodymium (iron-boron), samarium-cobalt or the like. In order to keep the magnetic field directed to the coil and not to influence other areas with the energy-detection magnet 5, the energy-detection magnet 5 is surrounded by a shield to be described at a later point. Thus, only one effective direction is achieved.

In other words, the activation-cycle counter 100 comprises the energy detection coil 4 arranged at one of a first and a second movable part of an at least two-part product or instrument and arranged to inductively detect generated energy, and the energy-generating magnet 5 arranged at the other one of the first and the second movable part of the at least two-part product or instrument and arranged to inductively generate energy to be detected by the energy detection coil 4.

That is, on one of the first and second movable parts, the part of the activation-cycle counter 100 comprising the components 1 to 4 and the housing 6 is arranged, and on the other one of the first and second movable parts, the energy-generating magnet 5 is arranged. The energy-generating magnet 5 and the energy detection coil 4 are arranged in such a way that, when the first and second movable parts move relative to each other, the energy-generating magnet 5 is movable in a detection range of the energy detection coil 4 relative to the latter and/or is guided past the latter and, in the process, induces energy in the energy detection coil 4 via the latter. This (here linear) relative movement is illustrated in FIG. 1 with an energy-generating magnet 5 indicated at various positions along a double arrow with a broken line and voltage arrow symbols pointing to the energy detection coil 4 at the various positions. It is understood that the relative movement is not limited to a linear movement, and that other relative movements are also suitable for generating the desired energy in the energy detection coil 4.

The memory and processing device 1 comprises a counter (not shown) and is arranged to detect a voltage signal based on the energy induced in the energy detection coil 4 at each relative movement and to increment the counter by one (1) at each detection of the voltage signal, thereby counting the number of executed activation cycles of the instrument.

The activation-cycle counter 100 with integrated energy detection coil 4 for detecting movement via the energy-generating magnet 5 is transferable and scalable to items, products, instruments and the like, provided that they have a certain two-part structure, i.e. at least a first movable part and a second movable part which are movable relative to each other.

FIG. 2 shows a schematic representation of the apparatus for detecting activation cycles of a hand-operated instrument according to a second configuration example.

While in the apparatus according to the first configuration example shown in FIG. 1, the magnetic core 2 is arranged with the coil 3 dedicated to it and the energy detection coil 4 is arranged separately from the magnetic core 2, in the second configuration example shown in FIG. 2, the energy detection coil is wound directly onto the magnetic core 2.

The magnetic core 2 with an associated coil is used in conjunction with a corresponding chip or combined component to provide RFID/NFC functionality, among other things. By winding the energy detection coil directly onto the magnetic core, a separate energy detection coil 4 can be advantageously omitted. In other words, the function of the separate energy detection coil 4 in the second configuration example is taken over by the coil 3 of the magnetic core 2.

The other components and the mode of operation of the second configuration example of the activation-cycle counter 100 shown in FIG. 2 correspond to the components and the mode of operation of the first configuration example of the activation-cycle counter 100 shown in FIG. 1, so that a redundant description in this respect can be conveniently omitted.

Preferably, the energy-generating magnet is a neodymium or a samarium-cobalt magnet and is surrounded by a shield in such a way that its magnetic field is directed in an effective direction 30 towards the energy detection coil and is attenuated in directions other than the effective direction. Advantageously, only one predeterminable effective direction is achieved.

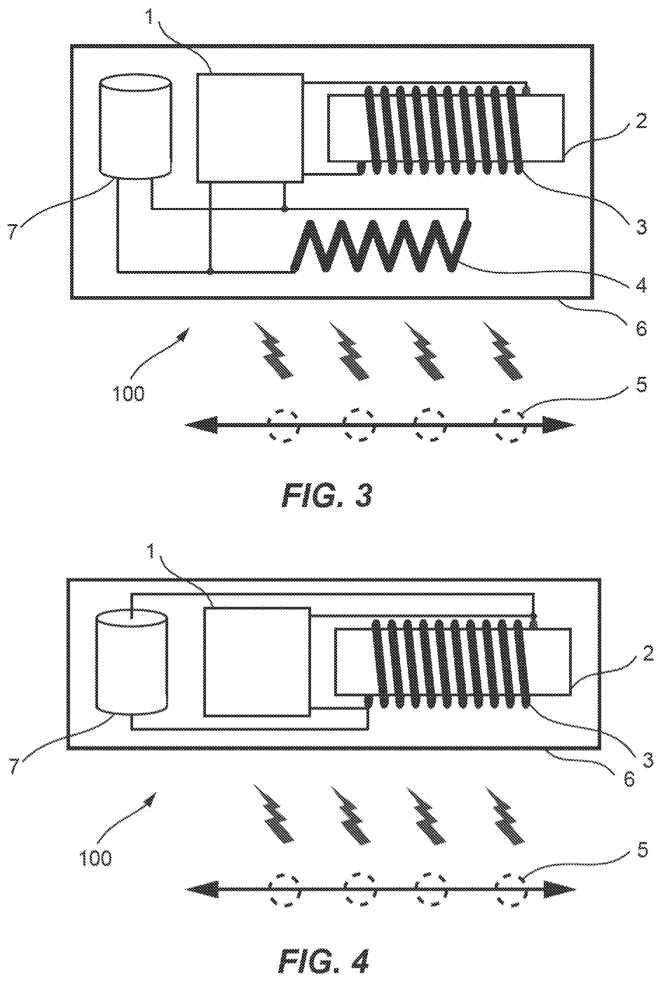

FIG. 3 shows a schematic representation of the apparatus for detecting activation cycles of a hand-operated instrument according to a third configuration example, and FIG. 4 shows a schematic representation of the apparatus for detecting activation cycles of a hand-operated instrument according to a fourth configuration example.

The third and fourth configuration examples each correspond in structure and mode of operation to the preceding first and second configuration examples, respectively, except that in the third and fourth configuration examples a smoothing buffer module 7 (which may include a capacitor, for example a capacitor serving as a variance, a PowerCap™ brand buffer module, a battery, etc.) is additionally arranged with charging electronics (not shown) to keep the memory and processing device (the chip) 1 ‘receivable’ for a longer time. The smoothing buffer module 7 thus forms an additional energy storage. In this context, the energy detection coil 3 may also be designed as an induction coil that enables inductive (wireless) energization of the smoothing buffer module 7. The required energy input can be provided by an inductive charger on an operating table, for example.

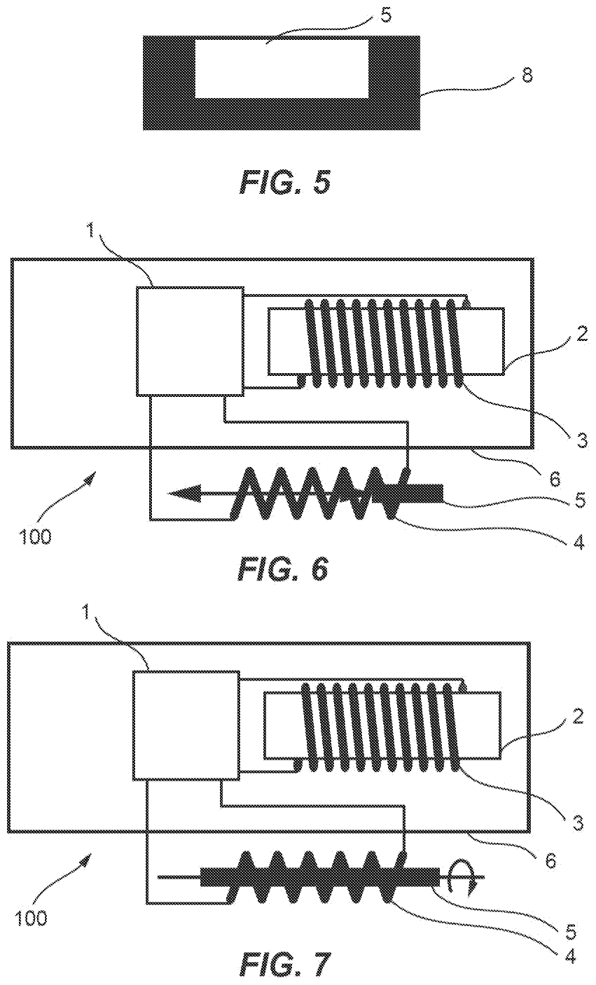

FIG. 5 shows a schematic representation of an energy-detection magnet 5 surrounded by a shield, which can be used as the energy-generating magnet 5 in configuration examples of the activation-cycle counter 100. In order to keep the magnetic field directed to the energy detection coil and not to influence other areas with the energy-generating magnet 5, the energy-generating magnet 5 is surrounded by a shield 8 in such a way that only one effective direction is achieved.

FIG. 6 shows a schematic representation of the apparatus for detecting activation cycles of a hand-operated instrument according to a fifth configuration example.

The fifth configuration example corresponds in structure and mode of operation to the preceding first configuration example, except that the energy detection coil 4 is arranged such that an energy-generating magnet inserted into the energy detection coil 5 induces energy by a linear (back and forth) movement in the energy detection coil 4.

FIG. 7 shows a schematic diagram of the apparatus for detecting activation cycles of a hand-operated instrument according to a sixth configuration example.

The sixth configuration example corresponds in structure and mode of operation to the preceding first configuration example, except that the energy detection coil 4 is designed in such a way that an energy-generating magnet 5 rotatably arranged in the energy detection coil 4 induces energy by a rotary movement. For adaptation to higher speeds, a transmission gearing (not shown) for higher speeds can also be arranged.

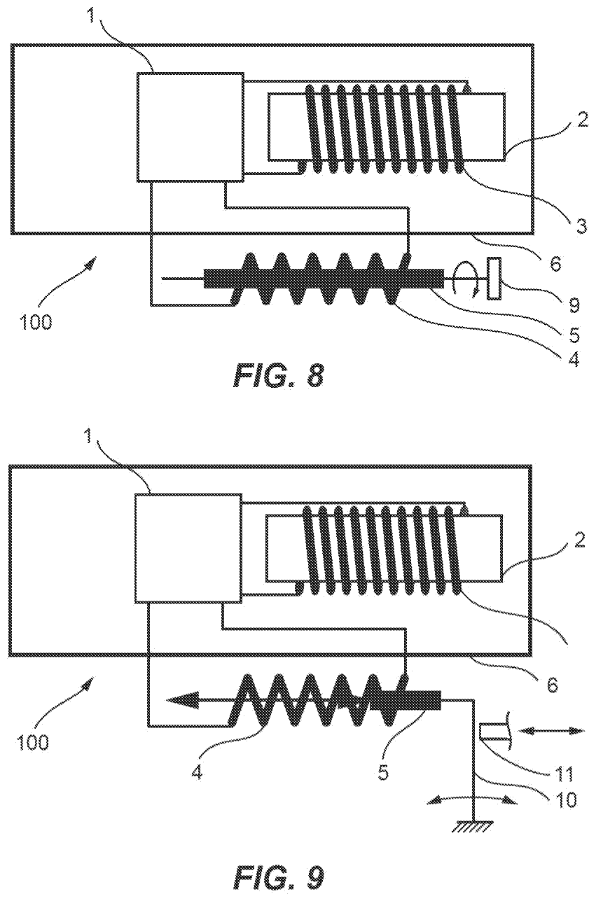

FIG. 8 shows a schematic representation of an apparatus for detecting activation cycles of a hand-operated instrument according to a seventh configuration example. The seventh configuration example corresponds in structure and mode of operation to the preceding sixth configuration example, except that in a modification thereof a flywheel mass 9, for example a flywheel, may additionally be arranged to maintain the rotary motion of the energy-generating magnet 5 for a longer time.

FIG. 9 shows a schematic representation of an apparatus for detecting activation cycles of a hand-operated instrument according to an eighth configuration example. In the eighth configuration example, in a further design, the energy detection coil 4 is arranged in such a way that an energy-detecting magnet 5 arranged in the energy detection coil 4 on a pivot arm 10 is caused to oscillate by a body 11 acting on, i.e. actuating, the pivot arm 10, wherein the body 11 may be in the form of a pin-shaped actuating element, for example, and can thereby generate energy in a prolonged manner.

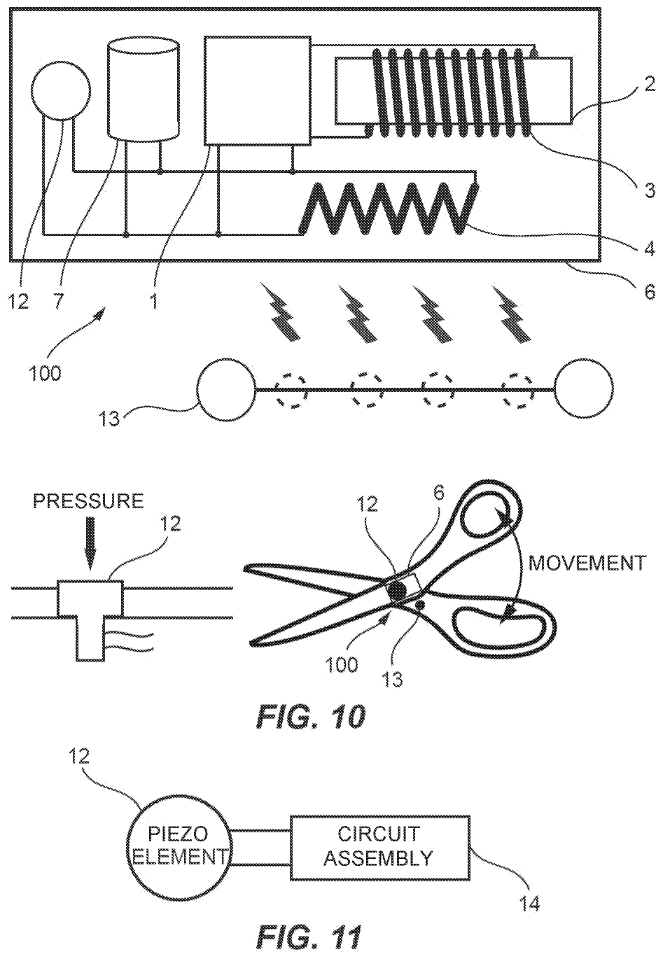

FIG. 10 shows a schematic representation of an apparatus for detecting activation cycles of a hand-operated instrument in an alternative embodiment according to a ninth configuration example.

In the alternative embodiment according to FIG. 10, a piezoelectric element or piezo element 12 is used as the energy generation device instead of the energy-generating magnet 5 and counting device, which builds up (electrical) voltage if a pressure-generating element 13 applies pressure, tension and/or torsion to it. Likewise, it can build up (electrical) voltage by the action of a vibration and a shock. Thus, due to pressure, tension, or torsion, an (electrical) voltage is generated when closing the (at least two-part) instrument via the relative movement of the first movable part and the second movable part thereof.

As shown in simplified form in the lower part of FIG. 10, the piezo element 12 may, for example, be arranged in the region of a pivot joint of an instrument (for example a pair of scissors) with two parts movable relative to each other on one of the two parts movable relative to one another, for example embedded in one of the legs of the scissors, and the pressure-generating element 13 may be arranged on the other one of the two parts movable relative to each other, for example embedded in the other one of the legs of the scissors. When the scissors are closed, the pressure-generating element 13 presses against the piezo element 12, which then builds up a voltage. This voltage is detected in the energy detection coil 4 and is converted into a signal that can be measured or further processed and ultimately counted by the memory and processing device 1.

In a further modification, for example, such a pivot joint may consist at least partially or entirely of a piezo element 12, which thus feeds the apparatus with energy due to pressure and torsion.

The piezo element 12 has to be operated with a suitable electrical or electronic circuit assembly 14 for energy organization or energy management. This circuit assembly 14 is shown schematically in FIG. 11 and is necessary to prevent the piezo element 12 from being energized by the smoothing and buffer module 7 (for example, by a capacitor contained therein that serves as a variance). Likewise, this circuit assembly 14 enables charging of such a capacitor.

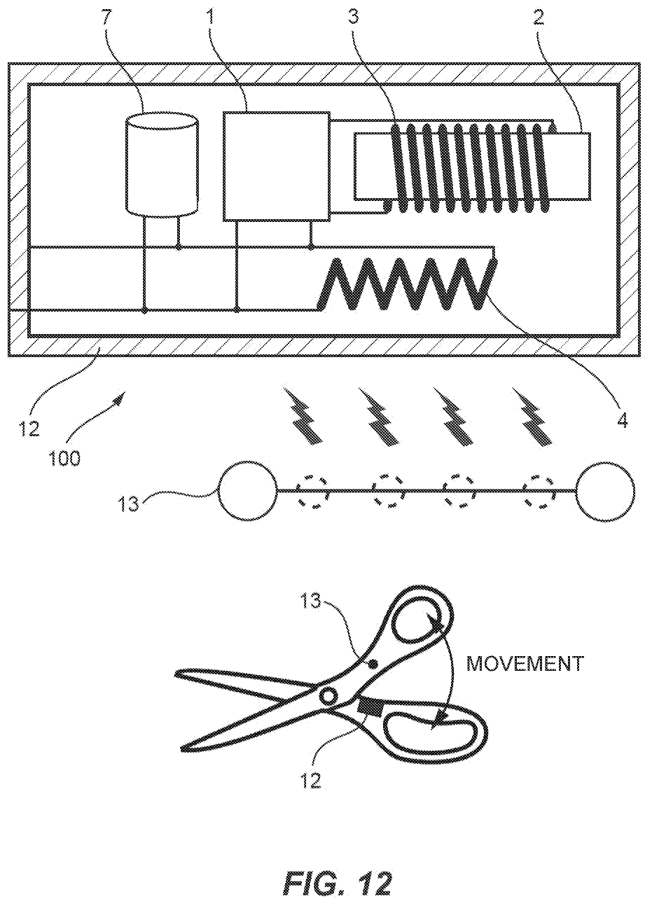

FIG. 12 a schematic representation of an apparatus for detecting activation cycles of a hand-operated instrument according to a tenth configuration example.

According to FIG. 12, in a further modification, the piezo element 12 is formed as a housing that tightly encloses the apparatus or assembly group as a housing and, when externally subjected to a vibration, shock or impact, or pressure, induces energy into the assembly group.

As described above, in an apparatus for detecting activation cycles of a hand-operated instrument in an activation cycle, a first and a second movable part of the instrument are movable relative to each other. An energy detection coil on one of the first and second movable parts detects inductively generated energy. An energy-generating magnet on the other one of the first and second movable parts inductively generates energy to be detected by the energy detection coil. The energy-generating magnet is movable relative to the energy detection coil when the first and second movable parts move relative to each other within a detection range of the energy detection coil and induces energy therein. A memory and processing device includes a counter, detects a voltage signal based on the induced energy at each relative movement, increments the counter by one at each detection of the voltage signal, and thereby counts the number of executed activation cycles of the instrument. In an alternative embodiment, the voltage signal is generated using a voltage built up by a piezoelectric element.

It is understood that the invention is not limited to the preceding configuration examples, but that changes, modifications, combinations and equivalent arrangements are readily available to a person skilled in the art within the scope of protection as defined in the claims.

1.-16. (canceled)

17. A hand-operated medical instrument comprising a first movable part and a second movable part, wherein the first movable part and the second movable part are movable relative to each other in an activation cycle, the hand-operated medical instrument further comprising an apparatus configured to detect activation cycles of the hand-operated medical instrument, the apparatus comprising:
an energy detection coil arranged on the first movable part and arranged to detect inductively generated energy;
an energy-generating magnet arranged on the second movable part and arranged to inductively generate energy to be detected by the energy detection coil; and
a memory and processing device having a counter and arranged to detect, at each relative movement, a voltage signal based on the energy induced in the energy detection coil and to increment the counter by one at each detection of the voltage signal, thereby counting the number of executed activation cycles of the hand-operated medical instrument,
wherein the energy-generating magnet and the energy detection coil are arranged such that, upon movement of the first movable part and the second movable part relative to each other, the energy-generating magnet is movable within a detection range of the energy detection coil relative thereto and induces energy therein via the energy detection coil.

18. The hand-operated medical instrument according to claim 17, wherein the hand-operated medical instrument is supplied with energy from an internal energy source.

19. The hand-operated medical instrument according to claim 17, wherein a magnetic core is provided which is attached to the apparatus and the energy detection coil is wound on the magnetic core.

20. The hand-operated medical instrument according to claim 17, wherein a magnetic core is provided which is attached to the apparatus, the energy detection coil is arranged separately from the magnetic core and the magnetic core has a further coil wound onto it.

21. The hand-operated medical instrument according to claim 20, wherein the energy-generating magnet is a neodymium or a samarium-cobalt magnet and is surrounded by a shield in such a way that its magnetic field is directed in an effective direction towards the energy detection coil and is attenuated in directions other than the effective direction.

22. The hand-operated medical instrument according to claim 21, wherein the energy-generating magnet is insertable into the energy detection coil and induces energy therein by a linear movement.

23. The hand-operated medical instrument according to claim 22, wherein the energy-generating magnet that can be introduced into the energy detection coil is arranged on a pivot arm and can be caused to oscillate by a body mechanically acting on the pivot arm.

24. The hand-operated medical instrument according to claim 21, wherein the energy-generating magnet is rotatably arranged in the energy detection coil and induces energy therein by a rotary movement.

25. The hand-operated medical instrument according to claim 24, wherein a transmission gearing is arranged to adjust the rotational speed of the rotary movement.

26. The hand-operated medical instrument according to claim 24, wherein a flywheel mass is arranged to assist in maintaining the rotary movement.

27. The hand-operated medical instrument according to claim 17, wherein the memory and processing device comprises an EEPROM and an integrated circuit or is formed as a combination component, and in conjunction with the magnetic core and the coil wound thereon provides an externally addressable and/or readable RFID/NFC apparatus with RFID/NFC functionality.

28. The hand-operated medical instrument according to claim 17, wherein a smoothing buffer module with charging electronics, wherein the smoothing buffer module comprises a capacitor, a PowerCap, and/or a battery and is provided to support the memory and processing device.

29. A hand-operated medical instrument comprising a first movable part and a second movable part, wherein the first movable part and the second movable part are movable relative to each other in an activation cycle, the hand-operated medical instrument further comprising an apparatus configured to detect activation cycles of the hand-operated medical instrument, the apparatus comprising:
a piezoelectric element arranged on the first movable part and arranged to be subjected to pressure, tension and/or torsion and to thereby create a voltage;
an electronic circuit assembly for operating the piezoelectric element;
a pressure-generating element arranged on the second movable part and arranged to subject the piezoelectric element to pressure, tension, and/or torsion;
an energy detection coil arranged to convert the voltage generated by the piezoelectric element into a detectable voltage signal; and
a memory and processing device comprising a counter and arranged to detect the voltage signal generated in the energy detection coil at each relative movement and to increment the counter by one at each detection of the voltage signal, thereby counting the number of executed activation cycles of the instrument,
wherein the piezoelectric element and the pressure-generating element are arranged in such a way that upon movement of the first movable part and the second movable part relative to each other, the pressure-generating element applies the pressure, tension, and/or torsion to the piezoelectric element and the voltage thereby generated by the piezoelectric element is applied to the energy detection coil and there generates the detectable voltage signal.

30. The hand-operated medical instrument according to claim 29, wherein a magnetic core is provided which is attached to the apparatus.

31. The hand-operated medical instrument according to claim 29, wherein the memory and processing device comprises an EEPROM and an integrated circuit or is formed as a combination component, and in conjunction with the magnetic core and the coil wound thereon provides an externally addressable and/or readable RFID/NFC apparatus with RFID/NFC functionality.

32. The hand-operated medical instrument according to claim 29, wherein a smoothing buffer module with charging electronics is provided, wherein the smoothing buffer module comprises a capacitor, a PowerCap, and/or a battery and is provided to support the memory and processing device.

33. The hand-operated medical instrument according to claim 29, wherein a pivot joint connecting the first movable part and the second movable part consists at least partially of a piezo element arranged to energize the apparatus via pressure and torsion.

34. The hand-operated medical instrument according to claim 29, wherein the piezo element is in the form of a housing and is arranged to tightly enclose the further components of the apparatus and to induce energy in the apparatus when subjected to vibration, shock or pressure.
